(19) United States
(12) Patent Application Publication
(10) Pub. No.: US 2002/0195104 A1
(43) Pub. Date: Dec. 26, 2002

(54) Apparatus for increasing the humidification of the air stream breathed by a patient

(76) Inventors: Massimo Fini, Mirandola (IT); Giovanni Mambrini, Concordia (IT)

Correspondence Address: FULWIDER PATTON LEE & UTECHT, LLP, HOWARD HUGHES CENTER, 6060 CENTER DRIVE, TENTH FLOOR, LOS ANGELES, CA, 90045, US

(21) Appl. No.: 10/147,298

(22) Filed: May 16, 2002

(30) Foreign Application Priority Data
May 22, 2001 (IT) MI2001A001073

(51) Int. Cl. A61M 15/00; H05B 3/00; A61M 16/00

(52) U.S. Cl. 128/203.17; 128/203.16

(57) ABSTRACT

An apparatus for increasing the humidification of the air stream breathed by a patient, having a tubular body that can be coupled to a heat and moisture exchange filter and forms a channel for the passage of an air stream. At least one portion of the channel is provided by a membrane impermeable to gases and permeable to ions and molecules with a partial charge. The membrane delimits an interspace that is external to the channel and allows passage of a water vapor flow.

### BACKGROUND OF THE INVENTION

1. Field of the Invention

The present invention relates to an apparatus for increasing the humidification of the air stream breathed by a patient.

2. Description of Related Art

It is known that the initial treatment of the air or gases inspired by a person normally occurs in the upper respiratory tract, i.e., in the so-called upper air tracts or ways; accordingly, in the case of patients subjected to mechanical ventilation with an endotracheal tube the contribution of the upper air tracts or ways is excluded completely and it is therefore necessary to humidify the air breathed by the patient.

This phenomenon is further exacerbated by the use of medical air, which due to technical requirements is particularly filtered and dry, with an absolute humidity of approximately 1 mg/l, thereby it is absolutely necessary to humidify the air stream in order to avoid the risk of causing lesions to the pulmonary epithelium of the patient, which would gradually worsen over time, becoming irreversible after a few hours.

In order to solve this problem, filtration devices for passive humidification, termed HME (Heat and Moisture Exchanger), are already known; they retain a significant fraction of the moisture and heat expired by the patient in order to return the moisture during inspiration.

This phenomenon has an efficiency of approximately 80% and offers no additional contribution in terms of humidity or heat with respect to what the patient exhales.

Devices are already known which allow to increase the humidification of the air, and for this purpose appropriate humidifiers are usually provided which in practice drip water onto the filtration element, which must be heated appropriately so as to facilitate the evaporation of the added water.

This approach causes considerable complications, since it requires connecting both a heating element and the dripping element to the filter.

### SUMMARY OF THE INVENTION

The aim of the present invention is to eliminate the drawbacks mentioned above, by providing an apparatus for increasing the humidification of the air stream breathed by a patient which also allows to provide an addition of heat without having to provide heat sources located proximate to the HME filter.

Within this aim, an object of the invention is to provide an apparatus that is particularly lightweight and therefore does not increase the weight of the apparatus to be connected to the patient.

Another object of the present invention is to provide an apparatus which, by way of its particular constructive characteristics, is capable of giving the greatest assurances of reliability and safety in use.

Another object of the present invention is to provide an apparatus that can be obtained easily starting from commonly commercially available elements and materials and is further competitive from a merely economical standpoint.

This aim and these and other objects that will become better apparent hereinafter are achieved by an apparatus for increasing the humidification of the air stream breathed by a patient, according to the invention, characterized in that it comprises a tubular body that can be coupled to a heat and moisture exchange filter and forms a channel for the passage of an air stream, at least one portion of the channel being provided by way of a membrane that is impermeable to gases and permeable to ions and molecules with a partial charge, the membrane delimiting an interspace that is external to the channel and allows passage of a water vapor flow.

### BRIEF DESCRIPTION OF THE DRAWINGS

Further characteristics and advantages of the present invention will become better apparent from the following detailed description of a preferred but not exclusive embodiment of an apparatus for increasing the humidification of the air stream breathed by a patient, illustrated only by way of non-limitative example in the accompanying drawings, wherein:

FIG. 1 is a schematic sectional view of the apparatus according to the invention;

FIG. 2 is a view of the ventilation unit to which the apparatus according to the invention is applied.

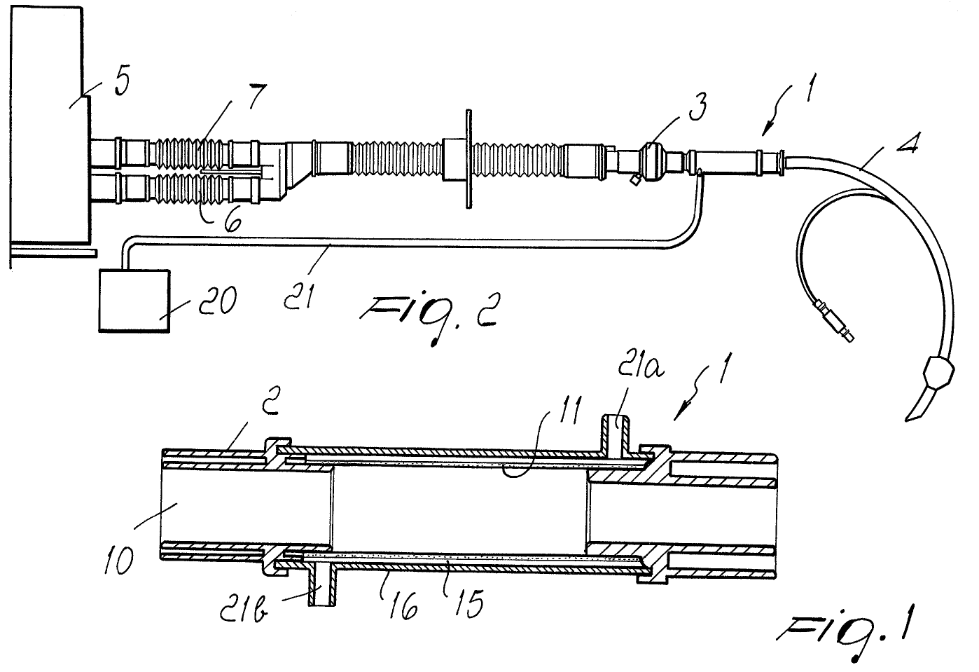

### DESCRIPTION OF THE PREFERRED EMBODIMENTS

With reference to the figures, the apparatus for increasing the humidification of the air stream breathed by a patient, according to a presently preferred embodiment of the invention, generally designated by the reference numeral 1, comprises a tubular body 2, which is interposed between the heat and moisture exchange filter 3, of a per se known type, and the endotracheal tube 4 that is connected to the patient.

The filter 3, as shown in FIG. 2, is arranged in a per se conventional manner downstream of the ventilation unit 5 from which the expiratory duct 6 and inspiratory duct 7 branch out.

In one presently preferred embodiment of the invention the tubular body 2 defines or forms internally a channel 10 for the passage of the respiratory air stream; at least one portion of the channel 10 is provided by a membrane 11 that is impermeable to gases and permeable to ions and molecules with a partial charge. Such membrane is provided by a tubular portion made of a copolymer of perfluorosulfonic acid and polytetrafluoroethylene in acid form; for example, a material of this kind is known commercially under the trademark Nafion by DuPont.

The membrane 11 delimits an interspace 15, in cooperation with an outer enclosure 16 provided on the tubular body 2, through which saturated-type water vapor flows; the membrane 11 allows the water to pass in the form of a molecule that is locked in a permanent dipole state and therefore has ionic characteristics in the molecular state, but does not allow the passage of all the other gases involved in respiration; accordingly, the Nafion membrane allows water to pass from a high-concentration environment to a lower-concentration environment until the intended equilibrium is reestablished.

The tubular portion made of Nafion can therefore increase the humidification of the air stream inside the channel 10 and also allows to heat the gases of the air stream, which accordingly reach the patient not only after being enriched with moisture but also after being duly heated.

In order to produce the stream of water vapor, there is a humidifier-heater 20, in which a heater acts as a source of vapor and a small centrifugal pump allows constant recycling of the warm saturated vapor by means of a double-lumen tube 21 that is connected to the inlet 21a and the outlet 21b, which are connected to the interspace 15.

With the apparatus described above it is therefore possible to provide continuous additional humidification of the air by using water vapor as a replacement of known devices, which required the use of a heating system inside the respiration duct and dripping on the filter.

From the above description it is evident that the invention achieves the intended aim and objects, and in particular the fact is stressed that an apparatus for increasing the humidification of the air stream breathed by a patient is provided which is particularly compact and easy to handle and allows, by way of the use of the semipermeable membrane made of Nafion, to achieve a passage of water while blocking the flow of gases through it; it should also be noted that the Nafion membrane is impermeable to viruses and bacteria.

The invention thus conceived is susceptible of numerous modifications and variations, all of which are within the scope of the appended claims.

All the details may further be replaced with other technically equivalent elements. In practice, the materials used, as well as the contingent shapes and dimensions, may be any of a variety of configurations, materials and dimensions satisfying the requirements of the invention.

The disclosures in Italian Patent Application No. MI2001A001073 from which this application claims priority are incorporated herein by reference.

What is claimed is:

1. An apparatus for increasing the humidification of the air stream breathed by a patient, comprising:
a heat and moisture exchange filter;
a tubular body connectable to said heat and moisture exchange filter;
a channel for passage of an air stream formed in said tubular body;
a membrane that is impermeable to gases and permeable to ions and molecules with a partial charge, said membrane forming at least part of said channel and delimiting an interspace that is external to said channel; and
a water vapor flow provided in said channel.

2. The apparatus of claim 1, wherein said tubular body is interposed between said heat and moisture exchange filter and a patient.

3. The apparatus of claim 1, wherein said membrane is provided as a tubular portion that is made of a copolymer of perfluorosulfonic acid and polytetrafluoroethylene in acid form.

4. The apparatus of claim 1, wherein said membrane is made of a commercially available copolymer of perfluorosulfonic acid and polytetrafluoroethylene in acid form.

5. The apparatus of claim 1, further comprising an outer enclosure formed on said tubular body, said interspace being delimited by said membrane and by said outer enclosure.

6. The apparatus of claim 5, further comprising a humidifier-heater for introducing saturated steam suitable to act both as a humidification element and as a heating element, said interspace being connected to said humidifier-heater.
